(19) United States
(12) Patent Application Publication
(10) Pub. No.: US 2024/0270725 A1
(43) Pub. Date: Aug. 15, 2024

(54) MAGL INHIBITORS

(71) Applicants: Hoffmann-La Roche Inc., Little Falls, NJ (US); Eidgenoessische Technische Hochschule Zuerich, Zuerich (CH)

(72) Inventors: Luca Claudio GOBBI, Buus (CH); Uwe Michael GRETHER, Efringen-Kirchen (DE); Yingfang HE, Zuerich (CH); Bernd KUHN, Reinach BL (CH); Linjing MU, Lenzburg (CH)

(73) Assignees: Hoffmann-La Roche Inc., Little Falls, NJ (US); Eidgenoessische Technische Hochschule Zuerich, Zuerich (CH)

(21) Appl. No.: 18/593,350

(22) Filed: Mar. 1, 2024

Related U.S. Application Data
(63) Continuation of application No. PCT/EP2022/074263, filed on Sep. 1, 2022.

(30) Foreign Application Priority Data
Sep. 3, 2021 (EP) 21194695.9

(51) Int. Cl. C07D 405/12; A61K 51/04

(52) U.S. Cl. CPC C07D 405/12; A61K 51/0459

(57) ABSTRACT

The invention provides new reversible monoacylglycerol lipase (MAGL) inhibitors that are useful for the treatment or prophylaxis of diseases or conditions associated with MAGL. The reversible MAGL inhibitors according to the present invention may also be labeled with radioisotopes and are thus useful for medical imaging, such as positron-emission tomography (PET) and/or autoradiography.

### CROSS REFERENCE TO RELATED APPLICATIONS

This application is a continuaiton of International Application No. PCT/EP2022/074263, filed Sep. 1, 2022, which claims benefit of priority to EP Application No. 21194695.9, filed Sep. 3, 2021, the disclosures of each of which are incorporated herein by reference in their entireties.

### FIELD OF THE INVENTION

The present invention relates to organic compounds useful for therapy or prophylaxis in a mammal, and in particular to monoacylglycerol lipase (MAGL) inhibitors for the treatment or prophylaxis of diseases or conditions associated with MAGL in a mammal. The present invention further relates to radiolabeled MAGL inhibitors useful for medical imaging, such as positron-emission tomography (PET) and/or autoradiography.

### BACKGROUND OF THE INVENTION

Monoacylglycerol lipase (MAGL) is a serine hydrolase that is highly expressed in the central nervous system (CNS) as well as in several peripheral organs (Karlsson M, Contreras J A, Hellman U, Tornqvist H, Holm C. cDNA Cloning, Tissue Distribution, and Identification of the Catalytic Triad of Monoglyceride Lipase. J Biol Chem. 1997;272:27218-27223; Dinh T P, Carpenter D, Leslie F M, et al. Brain monoglyceride lipase participating in endocannabinoid inactivation. Proc Natl Acad Sci. 2002;99:10819-10824). It plays an elemental role in the regulation of endocannabinoids and lipid levels in physiological and pathological conditions (Blankman J L, Simon G M, Cravat B F. A Comprehensive Profile of Brain Enzymes that Hydrolyze the Endocannabinoid 2-Arachidonoylglycerol. Chem Biol. 2007;14:1347-1356). Consequently, MAGL inhibitors are of great interest, and become potential therapeutic drug for the treatment against multiple diseases, including neurodegeneration, psychiatric disorders and cancer (Gil-Ordóñez A, Martín-Fontecha M, Ortega-Gutiérrez S, López-Rodríguez M L. Monoacylglycerol lipase (MAGL) as a promising therapeutic target. Biochem Pharmacol. 2018;157:18-32). Positron emission tomography (PET), as a non-invasive imaging technique, is supporting drug discovery and development by providing valuable information on drug-target engagement, accessing drug occupancy and monitoring treatment (Hou L, Rong J, Haider A, et al. Positron Emission Tomography Imaging of the Endocannabinoid System: Opportunities and Challenges in Radiotracer Development. 2020;6). Early developed MAGL radioligands are mainly irreversible ones such as [¹¹C]MA-PB (Ahamed M, Attili B, van Veghel D, et al. Synthesis and preclinical evaluation of [¹¹C]MA-PB-1 for in vivo imaging of brain monoacylglycerol lipase (MAGL). Eur J Med Chem. 2017;136:104-113), [¹¹C]SAR127303 (Wang L, Mori W, Cheng R, et al. Synthesis and Preclinical Evaluation of Sulfonamido-based [¹¹C-Carbonyl]-Carbamates and Ureas for Imaging Monoacylglycerol Lipase. Theranostics. 2016;6:1145-1159), [¹¹C]PF-06809247 (Zhang L, Butler C R, Maresca K P, et al. Identification and Development of an Irreversible Monoacylglycerol Lipase (MAGL) Positron Emission Tomography (PET) Radioligand with High Specificity. J Med Chem. 2019;62:8532-8543) and [¹⁸]PF-06795071 (Chen Z, Mori W, Fu H, et al. Design, Synthesis, and Evaluation of ¹⁸F-Labeled Monoacylglycerol Lipase Inhibitors as Novel Positron Emission Tomography Probes. J Med Chem. 2019;62:8866-8872), which could hardly provide comprehensive quantification of drug-target interaction in pharmacokinetic modeling (Hou L, Rong J, Haider A, et al. Positron Emission Tomography Imaging of the Endocannabinoid System: Opportunities and Challenges in Radiotracer Development. J Med Chem. 2021;64:123-149). [¹⁸F]T-401 currently stands for the most representative case as reversible MAGL PET radiotracer (Hattori Y, Aoyama K, Maeda J, et al. Design, Synthesis, and Evaluation of (4R)-1-{3-[2-(¹⁸F)Fluoro-4-methylpyridin-3-yl]phenyl}-4-[4-(1,3-thiazol-2-ylcarbonyl)piperazin-1-yl]pyrrolidin-2-one ([¹⁸F]T-401) as a Novel Positron-Emission Tomography Imaging Agent for Monoacylglycerol Lipas. J Med Chem. 2019;62:2362-2375). However, [¹⁸F]T-401 possesses a low brain uptake, with the presence of a CNS-penetrate radiometabolite. These characteristics impede MAGL visualization in the brain and complicate the quantification of specific-bound signals in kinetic modeling (Pike VW, PET radiotracers: crossing the blood-brain barrier and surviving metabolism. Trends Pharmacol Sci. 2009;30:431-440). Due to the lack of an appropriate reversible MAGL PET tracer, MAGL occupancy with therapeutic intervention or MAGL alteration under pathological condition have not been reported in precilinical stage so far. Taken together, there continues to be a need for reversible PET tracers to validate target engagement of therapeutic MAGL inhibitors, as well as to investigate MAGL expression levels under healthy and diseased conditions (Hou L, Rong J, Haider A, et al. Positron Emission Tomography Imaging of the Endocannabinoid System: Opportunities and Challenges in Radiotracer Development. J Med Chem. 2021;64:123-149).

### SUMMARY OF THE INVENTION

In a first aspect, the present invention provides a compound selected from the group consisting of

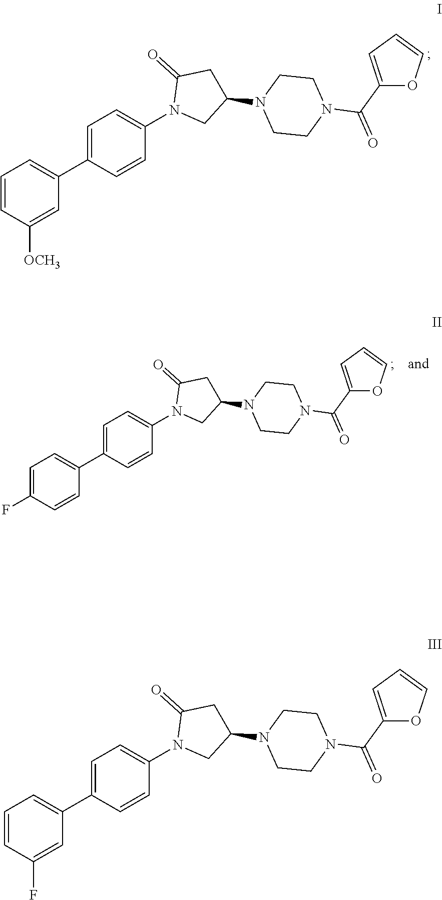 [SMILES: COc1cccc(-c2ccc(N3C[C@H](N4CCN(C(=O)c5ccco5)CC4)CC3=O)cc2)c1 ; O=C(c1ccco1)N1CCN([C@@H]2CC(=O)N(c3ccc(-c4ccc(F)cc4)cc3)C2)CC1 ; O=C(c1ccco1)N1CCN([C@@H]2CC(=O)N(c3ccc(-c4cccc(F)c4)cc3)C2)CC1]

or a pharmaceutically acceptable salt thereof, wherein said compound optionally comprises a radiolabel.

In a further aspect, the present invention provides a compound described herein for use as therapeutically active substance.

In a further aspect, the present invention provides a compound described herein for use in the treatment or prophylaxis of diseases or conditions associated with MAGL.

In a further aspect, the present invention provides a radiolabeled compound described herein for use in monoacylglycerol lipase (MAGL) occupancy studies.

In a further aspect, the present invention provides a radiolabeled compound described herein for use in diagnostic imaging of monoacylglycerol lipase (MAGL) in the brain of a mammal.

In a further aspect, the present invention provides a pharmaceutical composition comprising a radiolabeled compound described herein and a pharmaceutically acceptable carrier.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the in vitro autoradiograms of [¹¹C]-I (Cx=cortex; Hp=hippocampus; Cb=cerebellum; St=striatum; Th=thalamus) and MAGL mRNA expression in mouse brain retrieved from mouse.brain-map.org (experiment: 69015242).

FIG. 2 shows representative PET images averaged of [¹¹C]-I from 9.0 to 52.5 min in MAGL knockout (KO) and wild-type (WT) mice brains.

FIG. 3 shows time activity curves (TACs) of in the whole brain from [¹⁸F]-II and [¹⁸F]-III in MAGL KO and WT mice.

FIG. 4 shows the receptor occupancy by [¹⁸F]-II, which for every single injection was calculated by molar activity and included in saturation function. SUV_{0-90 min} was fitted to saturation function, and data were transferred to percentage receptor occupancy. ◯ means [¹⁸F]-II with PF-06795071; ● means [¹⁸F]-II alone (Baseline).

FIG. 5 shows an X-ray cocrystal structure of compound-II reversibly bound to human MAGL.

### DETAILED DESCRIPTION OF THE INVENTION

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like. In addition, these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like.

The abbreviation “MAGL” refers to the enzyme monoacylglycerol lipase. The terms “MAGL” and “monoacylglycerol lipase” are used herein interchangeably.

The term “mammal” includes humans, non-human primates such as chimpanzees and other apes and monkey species, farm animals such as cattle, horses, sheep, goats, and swine, domestic animals such as rabbits, dogs, and cats, laboratory animals including rodents, such as rats, mice, and guinea pigs. In certain embodiments, a mammal is a human. The term mammal does not denote a particular age or sex.

The terms “radiolabel” and “radioisotope” can be used interchangeably and refer to a radionuclide useful for PET imaging, such as ¹¹C, ¹³N, ¹⁵O, and ¹⁸F.

The terms “pharmaceutically acceptable excipient” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents or lubricants used in formulating pharmaceutical products.

The term “treatment” as used herein includes: (1) inhibiting the state, disorder or condition (e.g., arresting, reducing or delaying the development of the disease, or a relapse thereof in case of maintenance treatment, of at least one clinical or subclinical symptom thereof); and/or (2) relieving the condition (i.e., causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

The term “prophylaxis” as used herein includes: preventing or delaying the appearance of clinical symptoms of the state, disorder or condition developing in a mammal and especially a human that may be afflicted with or predisposed to the state, disorder or condition but does not yet experience or display clinical or subclinical symptoms of the state, disorder or condition.

The term “neuroinflammation” as used herein relates to acute and chronic inflammation of the nervous tissue, which is the main tissue component of the two parts of the nervous system; the brain and spinal cord of the central nervous system (CNS), and the branching peripheral nerves of the peripheral nervous system (PNS). Chronic neuroinflammation is associated with neurodegenerative diseases such as Alzheimer's disease, Parkinson's disease and multiple sclerosis. Acute neuroinflammation usually follows injury to the central nervous system immediately, e.g., as a result of traumatic brain injury (TBI).

The term “traumatic brain injury” (“TBI”, also known as “intracranial injury”), relates to damage to the brain resulting from external mechanical force, such as rapid acceleration or deceleration, impact, blast waves, or penetration by a projectile.

The term “neurodegenerative diseases” relates to diseases that are related to the progressive loss of structure or function of neurons, including death of neurons. Examples of neurodegenerative diseases include, but are not limited to, multiple sclerosis, Alzheimer's disease, Parkinson's disease and amyotrophic lateral sclerosis.

The term “mental disorders” (also called mental illnesses or psychiatric disorders) relates to behavioral or mental patterns that may cause suffering or a poor ability to function in life. Such features may be persistent, relapsing and remitting, or occur as a single episode. Examples of mental disorders include, but are not limited to, anxiety and depression.

The term “pain” relates to an unpleasant sensory and emotional experience associated with actual or potential tissue damage. Examples of pain include, but are not limited to, nociceptive pain, chronic pain (including idiopathic pain), neuropathic pain including chemotherapy induced neuropathy, phantom pain and phsychogenic pain. A particular example of pain is neuropathic pain, which is caused by damage or disease affecting any part of the nervous system involved in bodily feelings (i.e., the somatosensory system). In one embodiment, “pain” is neuropathic pain resulting from amputation or thoracotomy. In one embodiment, “pain” is chemotherapy induced neuropathy.

The term “neurotoxicity” relates to toxicity in the nervous system. It occurs when exposure to natural or artificial toxic substances (neurotoxins) alter the normal activity of the nervous system in such a way as to cause damage to nervous tissue. Examples of neurotoxicity include, but are not limited to, neurotoxicity resulting from exposure to substances used in chemotherapy, radiation treatment, drug therapies, drug abuse, and organ transplants, as well as exposure to heavy metals, certain foods and food additives, pesticides, industrial and/or cleaning solvents, cosmetics, and some naturally occurring substances.

The term “cancer” refers to a disease characterized by the presence of a neoplasm or tumor resulting from abnormal uncontrolled growth of cells (such cells being “cancer cells”). As used herein, the term cancer explicitly includes, but is not limited to, hepatocellular carcinoma, colon carcinogenesis and ovarian cancer.

### Compounds of the Invention

In one aspect, the present invention provides a compound selected from the group consisting of

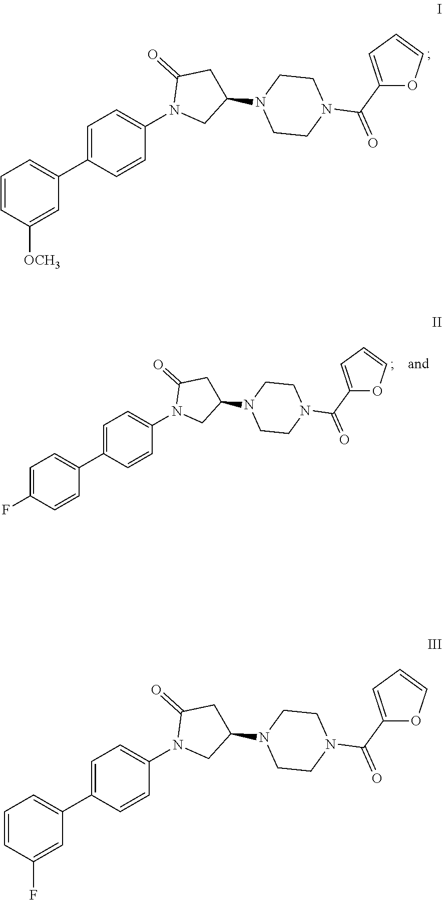 [SMILES: COc1cccc(-c2ccc(N3C[C@H](N4CCN(C(=O)c5ccco5)CC4)CC3=O)cc2)c1 ; O=C(c1ccco1)N1CCN([C@@H]2CC(=O)N(c3ccc(-c4ccc(F)cc4)cc3)C2)CC1 ; O=C(c1ccco1)N1CCN([C@@H]2CC(=O)N(c3ccc(-c4cccc(F)c4)cc3)C2)CC1]

or a pharmaceutically acceptable salt thereof, wherein said compound optionally comprises a radiolabel.

In one embodiment, said radiolabel is selected from ¹¹C, ¹³N, ¹⁵O, and ¹⁸F.

In a preferred embodiment, said radiolabel is selected from ¹¹C and ¹⁸F.

In a preferred embodiment, the present invention provides a radiolabeled compound selected from the group consisting of

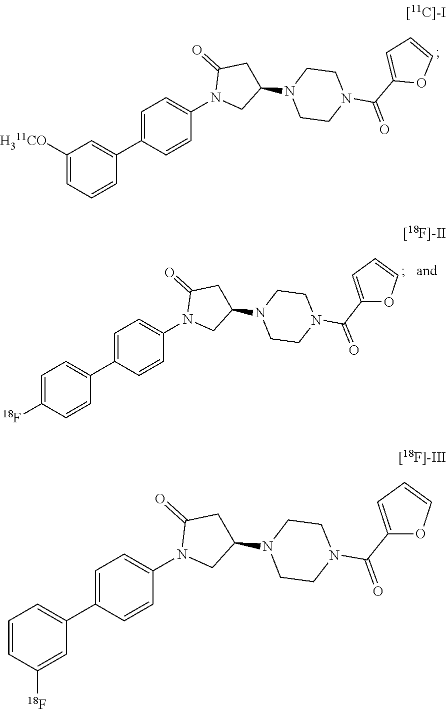

or a pharmaceutically acceptable salt thereof.

In one embodiment, the radiolabeled compound according to the invention is

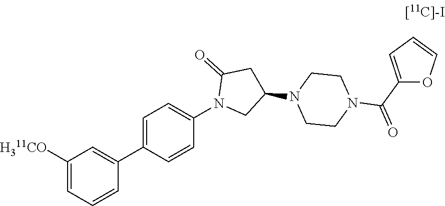

or a pharmaceutically acceptable salt thereof.

In one embodiment, the radiolabeled compound according to the invention is

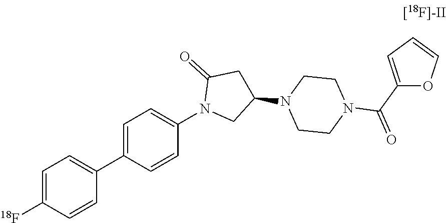 [SMILES: O=C(c1ccco1)N1CCN([C@@H]2CC(=O)N(c3ccc(-c4ccc([18F])cc4)cc3)C2)CC1]

or a pharmaceutically acceptable salt thereof.

In one embodiment, the radiolabeled compound according to the invention is

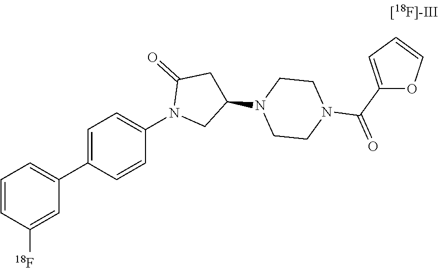 [SMILES: O=C(c1ccco1)N1CCN([C@@H]2CC(=O)N(c3ccc(-c4cccc([18F])c4)cc3)C2)CC1]

or a pharmaceutically acceptable salt thereof.

### Using the Compounds of the Invention

The compounds of the present invention are potent reversible MAGL inhibitors that may be used for the treatment or prophylaxis of diseases or conditions associated with MAGL. Examplary diseases or conditions that may be associated with MAGL are neuroinflammation, neurodegenerative diseases, pain, cancer, mental disorders, multiple sclerosis, Alzheimer's disease, Parkinson's disease, amyotrophic lateral sclerosis, traumatic brain injury, neurotoxicity, stroke, epilepsy, anxiety, migraine, depression, inflammatory bowel disease, abdominal pain, abdominal pain associated with irritable bowel syndrome and/or visceral pain.

Hence, in one aspect, the present invention provides a method of treatment or prophylaxis of neuroinflammation, neurodegenerative diseases, pain, cancer, mental disorders, multiple sclerosis, Alzheimer's disease, Parkinson's disease, amyotrophic lateral sclerosis, traumatic brain injury, neurotoxicity, stroke, epilepsy, anxiety, migraine, depression, inflammatory bowel disease, abdominal pain, abdominal pain associated with irritable bowel syndrome and/or visceral pain in a mammal, said method comprising administering to said mammal a therapeutically active amount of a compound of formula I, II, or III, or a pharmaceutically acceptable salt thereof.

In a further aspect, the present invention provides a compound of formula I, II, or III, or a pharmaceutically acceptable salt thereof, for use in a method of treatment or prophylaxis described herein.

In a further aspect, the present invention provides the use of a compound of formula I, II, or III, or a pharmaceutically acceptable salt thereof, in a method of treatment or prophylaxis described herein.

In a further aspect, the present invention provides the use of a compound of formula I, II, or III, or a pharmaceutically acceptable salt thereof, for the manufacture of a medicament for the treatment or prophylaxis of neuroinflammation, neurodegenerative diseases, pain, cancer, mental disorders, multiple sclerosis, Alzheimer's disease, Parkinson's disease, amyotrophic lateral sclerosis, traumatic brain injury, neurotoxicity, stroke, epilepsy, anxiety, migraine, depression, inflammatory bowel disease, abdominal pain, abdominal pain associated with irritable bowel syndrome and/or visceral pain.

The compounds of the present invention may be radiolabeled and used, for example, as non-covalent, reversible PET tracers to validate target engagement of therapeutic MAGL inhibitors, as well as to investigate MAGL levels under normal and disease conditions.

Thus, in one aspect, the present invention provides a method of diagnostic imaging of monoacylglycerol lipase (MAGL) in the brain of a mammal, comprising:
(a) administering to the mammal a detectable quantity of a radiolabeled compound described herein, or of a pharmaceutical composition comprising a radiolabeled compound described herein; and
(b) detecting the radiolabeled compound when associated with MAGL.

In a further aspect, the present invention provides a radiolabeled compound as described herein, or a pharmaceutically acceptable salt thereof, or a pharmaceutical composition comprising a radiolabeled compound described herein, for use in monoacylglycerol lipase (MAGL) occupancy studies.

In one embodiment, said monoacylglycerol lipase (MAGL) occupancy studies comprises contacting MAGL with a radiolabeled compound disclosed herein, or a pharmaceutically acceptable salt thereof.

In a further aspect, the present invention provides a radiolabeled compound as described herein, or a pharmaceutically acceptable salt thereof, or a pharmaceutical composition comprising a radiolabeled compound described herein for use in a method of diagnostic imaging of monoacylglycerol lipase (MAGL) in the brain of a mammal.

In a further aspect, the present invention provides the use of a radiolabeled compound as described herein, or a pharmaceutically acceptable salt thereof, or a pharmaceutical composition comprising a radiolabeled compound described herein, in a method of diagnostic imaging of monoacylglycerol lipase (MAGL) in the brain of a mammal.

In a further aspect, the present invention provides the use of a radiolabeled compound as described herein, or of a pharmaceutically acceptable salt thereof, for the preparation of a medicament for the diagnostic imaging of monoacylglycerol lipase (MAGL) in the brain of a mammal.

In one embodiment, said diagnostic imaging is positron-emission tomography (PET).

In one embodiment, said diagnostic imaging of monoacylglycerol lipase (MAGL) in the brain of a mammal comprises contacting monoacylglycerol lipase (MAGL) with a radiolabeled compound disclosed herein, or a pharmaceutically acceptable salt thereof.

### EXAMPLES

### Example 1—Synthesis of Intermediates 1-4

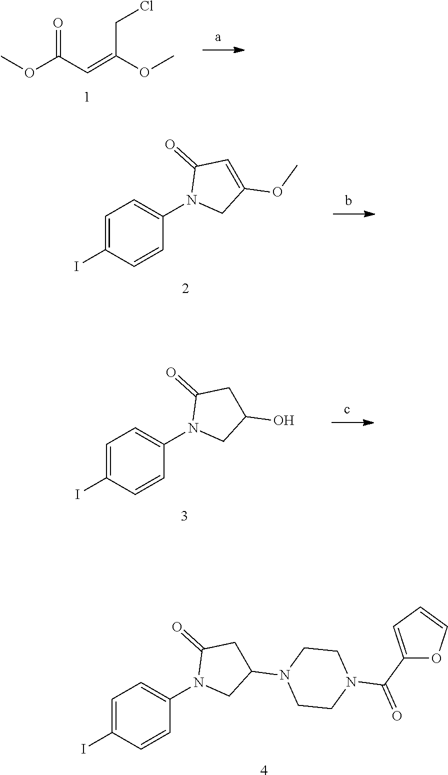

Scheme 1. Synthesis of intermediates 2-4. a) triethylamine, tetrabutylammonium iodide, 3-iodoaniline, CH₃CN/toluene, 50° C., overnight, 24%; b) conc. hydrocholoride acid, CH₃CN, 50° C., 4-5 h, 85%; c) sodium cyanoborohydride, furan-2-yl(piperazin-1-yl)methanone, acetic acid, anhydrous THF, r.t, overnight, 49%.
Step a—Synthesis of 1-(4-Iodophenyl)-4-methoxy-1,5-dihydro-2H-pyrrol-2-one (2).

Compound 1 (4.12 g, 25.0 mmol), 4-iodoaniline (4.00 g, 18.3 mmol), tetrabutylammonium iodide (67.5 mg, 0.18 mmol), and triethylamine (2.80 mL, 20.1 mmol) were dissolved in acetonitrile (15 mL). After stirring in an ice bath for 10 min, the reaction mixture was heated at 50° C. for 14 h. The reaction was cooled down to room temperature, and 1 M HCl was added to adjust pH to 3. The resulting mixture was diluted with water and extracted with ethyl acetate. The combined organic layers were washed with brine, dried over MgSO₄, and concentrated under reduced pressure. To the residue, toluene (10 mL) and acetic acid (1 mL) were added, and the mixture was heated at 50° C. for 4 h. The reaction was concentrated, and the product crystalized with methanol/diisopropyl ether (1:1). The desired product was obtained as a white powder (1.46 g, 25%) after washing with diisopropyl ether. ¹H NMR (400 MHz, Chloroform-d) δ 7.61 (d, J=9.0 Hz, 2H), 7.42 (d, J=9.0 Hz, 2H), 5.16 (s, 1H), 4.21 (s, 2H), 3.85 (s, 3H). HRMS (ESI) calculated for C₁₁H₁₁INO₂⁺ [M+H]⁺ 315.9829 m/z, found 315.9828 m/z.

Step b—Synthesis of 4-Hydroxy-1-(4-iodophenyl)-1,5-dihydro-2H-pyrrol-2-one (3).

Compound 2 (1.30 g, 4.14 mmol) was dissolved in 10 mL acetonitrile with 6 mL concentrated HCl. Upon consumption of 2 as monitored by LC-MS, the solvent was removed under reduced pressure. The resulting residue was diluted with water, and extracted with ethyl acetate. The combined organic layers were dried over MgSO₄, and concentrated. The crude was directly applied in the next step without any further purification.

Step c—Synthesis of 4-(4-(Furan-2-carbonyl)piperazin-1-yl)-1-(4-iodophenyl)pyrrolidin-2-one (4).

Compound 3 (570 mg, 1.89 mmol) and furan-2-yl(piperazin-1-yl)methaone (409 mg, 2.27 mmol) in 10 mL anhydrous THF were stirred at room temperature for 10 min. Acetic acid (217 μL, 3.8 mmol) was then added dropwise with subsequently sodium cyanoborohydride (357 mg, 5.68 mmol). The reaction mixture was stirred at room temperature under nitrogen protection for 22 h. Water was added to quench the reaction, and the resulting mixture was extracted with EtOAc, and dried over MgSO₄. After filtration, the solvent was removed under reduced pressure. Flash column chromatography was carried out with EtOAc to afford the title compound as a white powder (523 mg, 59%). ¹H NMR (400 MHz, Chloroform-d) δ 7.65 (d, J=8.9 Hz, 2H), 7.47 (s, 1H), 7.36 (d, J=8.9 Hz, 2H), 7.01 (d, J=3.3 Hz, 1H), 6.47 (dd, J=3.3, 1.7 Hz, 1H), 3.91-3.72 (m, 6H), 3.24 (p, J=7.8 Hz, 1H), 2.81-2.68 (m, 1H), 2.67-2.50 (m, 5H). HRMS (ESI) calculated for C₁₉H₂₁IN₃O₃⁺ [M+H]⁺ 466.0622 m/z, found 466.0628 m/z.

### Example 2—Synthesis of (R)-4-(4-(Furan-2-carbonyl)piperazin-1-yl)-1-(3′-methoxy-[1,1′-biphenyl]-4-yl)pyrrolidin-2-one (Compound (I))

(3-methoxylphenyl) boronic acid (43.0 mg, 0.28 mmol), compound 4 (120 mg, 0.26 mmol), cesium carbonate (210 mg, 0.65 mmol), tris(dibenzylideneacetone)dipalladium (0) (23.6 mg, 0.03 mmol) and Sphos (21.2 mg, 0.05 mmol) were added to a two-necked flask. The system was then closed, and filled in with nitrogen. 5 mL DMF previously purged with nitrogen was added to the flask, and the resulting mixture was heated under reflux at 85° C. overnight. Upon consumption of compound 4, the reaction was cooled down to room temperature, filtrated and concentrated. The crude material was purified by semi-preparative HPLC to afford the desired product as a white powder (87 mg, 76%). Chiral separation was carried out using supercritical fluid chromatography (SFC) to isolate the title compound. ¹H NMR (400 MHz, Chloroform-d) δ 7.62 (s, 4H), 7.51 (dd, J=1.8, 0.8 Hz, 1H), 7.39-7.32 (m, 1H), 7.18-7.13 (m, 2H), 7.11-7.08 (m, 1H), 6.93-6.88 (m, 1H), 6.53 (dd, J=3.5, 1.8 Hz, 1H), 4.30 (dd, J=11.1, 5.1 Hz, 1H), 4.26-4.05 (m, 5H), 3.98-3.89 (m, 1H), 3.87 (s, 3H), 3.19-3.05 (m, 4H), 2.98 (d, J=7.3 Hz, 2H). HRMS (ESI) calculated for C₂₆H₂₇N₃NaO₄⁺ [M+Na] +468.1894 m/z, found 468.1894 m/z.

### Example 3—Synthesis of (R)-1-(3′-Fluoro-[1,1′-biphenyl]-4-yl)-4-(4-(furan-2-carbonyl)piperazin-1-yl)pyrrolidin-2-one (Compound (III))

The procedure described for the synthesis of Compound (I) was applied to 3-fluorobenzeneboronic acid (57.9 mg, 0.41 mmol), compound 4 (175 mg, 0.38 mmol), cesium carbonate (307 mg, 0.94 mmol), tris(dibenzylideneacetone)dipalladium (0) (34.5 mg, 0.04 mmol) and Sphos (31 mg, 0.08 mmol) to afford the title compound as a white powder after chiral separation. ¹H NMR (400 MHz, Chloroform-d) δ 7.66-7.62 (m, 2H), 7.61-7.58 (m, 2H), 7.52-7.50 (m, 1H), 7.44-7.32 (m, 2H), 7.28-7.23 (m overlapped with chloroform-d, 1H), 7.16 (dd, J=3.6, 0.9 Hz, 1H), 7.08-7.01 (m, 1H), 6.54 (dd, J=3.5, 1.8 Hz, 1H), 4.36 (dd, J=11.2, 5.0 Hz, 1H), 4.25-4.13 (m, 5H), 4.08-3.97 (m, 1H), 3.31-3.10 (m, 4H), 3.06-3.00 (m, 2H). HRMS (ESI) calculated for C₂₅H₂₄FN₃NaO₃⁺ [M+Na]⁺ 456.1694 m/z, found 456.1699 m/z.

### Example 4—Synthesis of (R)-1-(4′-Fluoro-[1,1′-biphenyl]-4-yl)-4-(4-(furan-2-carbonyl)piperazin-1-yl)pyrrolidin-2-one (Compound (II))

The procedure described for the synthesis of Compound (I) was applied to 4-fluorobenzeneboronic acid (33.1 mg, 0.24 mmol), compound 4 (100 mg, 0.22 mmol), cesium carbonate (175 mg, 0.54 mmol), tris(dibenzylideneacetone)dipalladium (0) (19.7 mg, 0.02 mmol) and Sphos (17.7 mg, 0.04 mmol) to afford the title compound as a white powder after chiral separation. ¹H NMR (400 MHz, Chloroform-d) δ 7.64 (d, J=8.7 Hz, 2H), 7.58 (d, J=8.7 Hz, 2H), 7.55-7.51 (m, 3H), 7.17-7.12 (m, 3H), 6.55 (dd, J=3.5, 1.8 Hz, 1H), 4.32 (dd, J=11.0, 5.1 Hz, 1H), 4.26-4.02 (m, 5H), 3.96 (p, J=7.3 Hz, 1H), 3.14 (br, 4H), 3.05-2.97 (m, 2H). HRMS (ESI) calculated for C₂₅H₂₅FN₃O₃⁺ [M+H]⁺ 434.1874 m/z, found 434.1875 m/z.

### Example 5—Synthesis of [¹¹C]-I

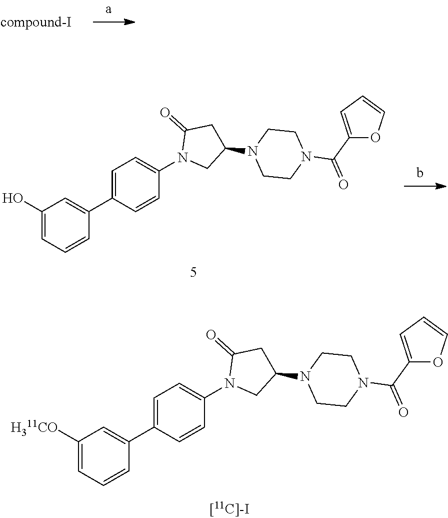

Scheme 2. Synthesis of precursors 5 and [¹¹C]-I. a) BBr₃, anhydrous DCM, 0° C., overnight; b) [¹¹C]CH₃I, Cs₂CO₃, 5 min, anhydrous DMF, 90° C.
Step a—Synthesis of (R)-4-(4-(Furan-2-carbonyl)piperazin-1-yl)-1-(3′-hydroxy-[1,1′-biphenyl]-4-yl)pyrrolidin-2-one (5).

To a solution of Compound (I) (107 mg, 0.241 mmol) in 5 mL anhydrous dichloromethane, 1 M boron tribromide in dichloromethane (1.90 mL, 1.93 mmol) was added dropwise at 0° C. The reaction mixture was then stirred at room temperature upon the consumption of the reagent. Saturated NaHCO₃ solution was added to quench the reaction, and the resulting mixture was extracted with EtOAc. The combined organic phase was dried over MgSO₄, filtrated, concentrated and purified by flash column chromatography (Silica gel, EtOH/CHCl₁₃=1/35−1/20). The title compound was obtained as white solid (54 mg, 75%). Chiral separation was carried out using supercritical fluid chromatography (SFC) to isolate 5 as R-isoform. ¹H NMR (400 MHz, Acetone-d₆) δ 7.83 (d, J=8.8 Hz, 2H), 7.71-7.68 (m, 1H), 7.63 (d, J=8.8 Hz, 2H), 7.28 (t, J=8.1 Hz, 1H), 7.15-7.10 (m, 2H), 6.97 (dd, J=3.4, 0.7 Hz, 1H), 6.83 (ddd, J=8.1, 2.2, 1.0 Hz, 1H), 6.58 (dd, J=3.4, 1.8 Hz, 1H), 4.11 (dd, J=9.7, 7.4 Hz, 1H), 3.89 (dd, J=9.7, 6.6 Hz, 1H), 3.80 (br, 4H), 3.41-3.30 (m, 1H), 2.76 (dd, J=16.6, 8.1 Hz, 1H), 2.70-2.55 (m, 5H). HRMS (ESI) calculated for C₂₅H₂₆N₃O₄⁺ [M+H]⁺ 432.1918 m/z, found 432.1920 m/z.

Step b—Synthesis of (R)-4-(4-(furan-2-carbonyl)piperazin-1-yl)-1-(3′-(methoxy-¹¹C)-[1,1′-biphenyl]-4-yl)pyrrolidin-2- one ([¹¹C]-(I))

[¹¹C]CO₂ was produced via the ¹⁴N(p,α)¹¹C nuclear reaction by bombardment of a nitrogen gas target fortified with 0.5% oxygen using a Cyclone 18/9 cyclotron (18 MeV; IBA, Belgium). [¹¹C]CH₄ was afterwards obtained by reduction of [¹¹C]CO₂ via nickel catalyst, and gas phase iodination was employed to generate [¹¹C]CH₃I. The resulting [¹¹C]CH₃I was bubbled into the reaction vial with 5 mg Cs₂CO₃ and 0.5 mg phenol precursor (1 mg/mL in anhydrous DMF, 0.5 mL). The mixture was then heating at 90° C. for 3 min. After dilution with 1.6 mL water, the reaction mixture was loaded to a semi-preparative HPLC for purification. The collected fraction was diluted with 8 mL Milli-Q water, passed through a pre-conditioned C18 light cartridge (Waters, WAT023501), and subsequently washed with 5 mL Milli-Q water. After elution with 0.5 mL EtOH, the final radioligand was formulated with phosphate-buffered saline (9.5 mL, Gibco) to give a neutralized solution. The identity of the radiotracer was confirmed by co-injection with Compound (I) in analytical HPLC, and the radiochemical purity was greater than 99%.

### Example 6—Synthesis of [¹⁸F]-II and [¹⁸F]-III

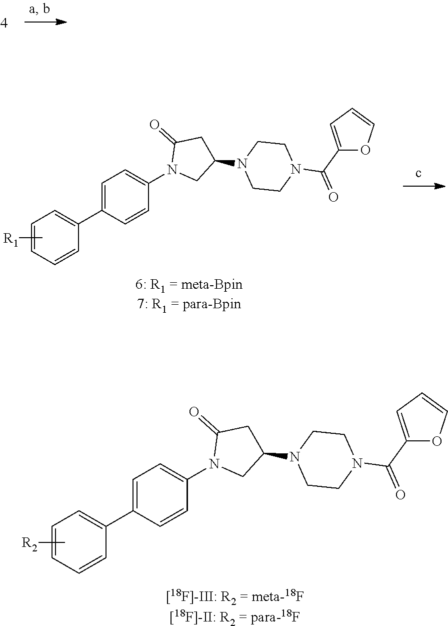

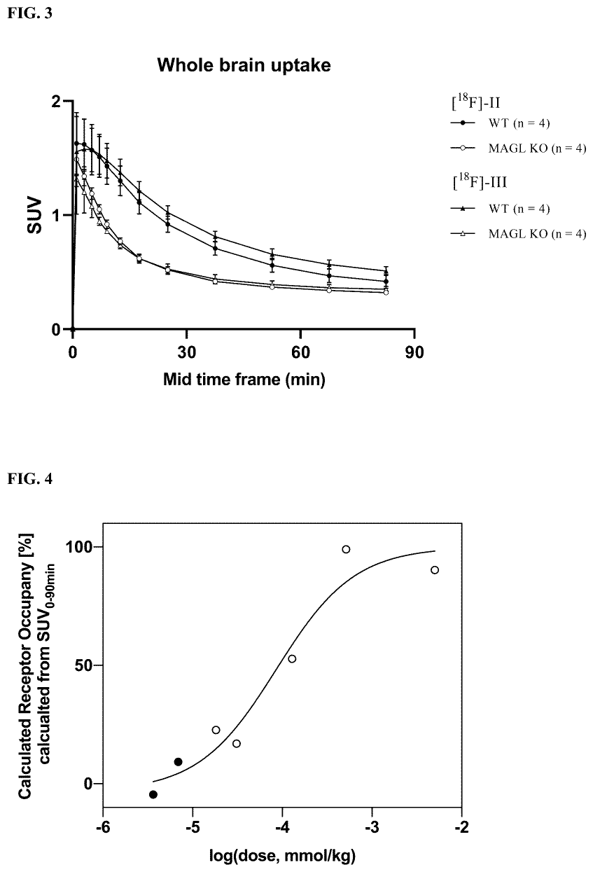

FIG. 3. Syntheses of precursor 6 and 7, and radiosyntheses of [¹⁸F]-II and [¹⁸F]-III. a) 1,4-phenyldiboronic acid, potassium acetate, [1,1′-bis(diphenylphosphino)ferrocene]dichloropalladium, anhydrous DMF, 80° C., 4 h, for 6, 1,3-bis(4,4,5,5-tetramethyl-1,3,2-dioxaborolan-2-yl)benzene; for 7, 1,4-bis(4,4,5,5-tetramethyl-1,3,2-dioxaborolan-2-yl)benzene; b) Chiral supercritical fluid chromatography separation; c) 6 or 7, [¹⁸F]F⁻, Kryptofix® 222, K₂C₂O₄, K₂CO₃ and Cu(OTf)₂Py₄, DMA/n-BuOH=2/1, 110° C., 10 min.
Steps a,b—Synthesis of (R)-4-(4-(Furan-2-carbonyl)piperazin-1-yl)-1-(3′-(4,4,5,5-tetramethyl-1,3,2-dioxaborolan-2-yl)-[1,1′-biphenyl]-4-yl)pyrrolidin-2-one (6).

[1,1′-Bis(diphenylphosphino)ferrocene]dichloropalladium (7.9 mg, 0.011 mmol), potassium acetate (63.3 mg, 0.84 mmol) and 1,3-bis(4,4,5,5-tetramethyl-1,3,2-dioxaborolan-2-yl)benzene (142 mg, 0.43 mmol) was added to a two-necked flask under nitrogen protection. Compound 4 (100 mg, 0.22 mmol) dissolved in 5 mL anhydrous DMF was added to the flask in one portion, and the resulting mixture was later heated at 85° C. Upon the consumption of compound 4, water was added to quench the reaction. The mixture was extracted with EtOAc, and the combined organic layers were dried over MgSO₄, filtrated, and concentrated. The residue was purified by flash column chromatography (Silica gel, EtOAc), affording the title compound as brown powder (47 mg, 41%). Chiral separation was carried out using supercritical fluid chromatography (SFC) to isolate 6 as R-isomer: ¹H NMR (400 MHz, Chloroform-d) δ 8.02 (s, 1H), 7.78 (d, J=7.4 Hz, 1H), 7.70-7.60 (m, 5FH), 7.50-7.41 (m, 2H), 7.07-6.99 (m, 1H), 6.54-6.43 (m, 1H), 4.13-3.65 (m, 6H), 3.29 (p, J=7.6 Hz, 1H), 2.79 (dd, J=16.7, 8.0 Hz, 1H), 2.72-2.40 (m, 5H), 1.36 (s, 12H). HRMS (ESI) calculated for C₃₁H₃₇BN₃O₅⁺ [M+H]⁺ 542.2826 m/z, found 542.2828 m/z.

Steps a,b—Synthesis of (R)-4-(4-(Furan-2-carbonyl)piperazin-1-yl)-1-(4′-(4,4,5,5-tetramethyl-1,3,2-dioxaborolan-2-yl)-[1,1′-biphenyl]-4-yl)pyrr olidin-2-one (7).

[1,1′-Bis(diphenylphosphino)ferrocene]dichloropalladium (10 mg, 0.014 mmol), potassium acetate (82 mg, 0.84 mmol) and 1,4-bis(4,4,5,5-tetramethyl-1,3,2-dioxaborolan-2-yl)benzene (185 mg, 0.56 mmol) was added to a two-necked flask under nitrogen protection. Compound 4 (130 mg, 0.28 mmol) dissolved in 5 mL anhydrous DMF was added to the flask in one portion, and the resulting mixture was later heated at 85° C. Upon the consumption of compound 4, water was added to quench the reaction. The mixture was extracted with EtOAc, and the combined organic layers were dried over MgSO₄, filtrated, and concentrated. The residue was purified by flash column chromatography (Silica gel, EtOAc), affording the title compound as brown powder (148 mg, 49%). Chiral separation was carried out using supercritical fluid chromatography (SFC) to isolate 7 as R-isomer: ¹H NMR (400 MHz, Chloroform-d) δ 7.87 (d, J=7.8 Hz, 2H), 7.73-7.61 (m, 4H), 7.59 (d, J=8.0 Hz, 2H), 7.48 (s, 1H), 7.03 (d, J=3.3 Hz, 1H), 6.49 (dd, J=3.3, 1.7 Hz, 1H), 4.04-3.94 (m, 1H), 3.92-3.72 (m, 5H), 3.30 (p, J=7.5 Hz, 1H), 2.80 (dd, J=16.7, 8.1 Hz, 1H), 2.73-2.54 (m, 5H), 1.36 (s, 12H). HRMS (ESI) calculated for C₃₁H₃₇BN₃O₅⁺ [M+H]⁺ 542.2826 m/z, found 542.2828 m/z.

Step c—Synthesis of (R)-1-(3′-(fluoro-¹⁸F)-[1,1′-biphenyl]-4-yl)-4-(4-furan-2-carbonyl)piperazin-1-yl)pyrrolidin-2-one ([¹⁸F]-III)

[¹⁸F]fluoride ions were produced by bombardment of 98% enriched ¹⁸O-water via the ¹⁸O(p,n)¹⁸F nuclear reaction. The aqueous solution was transferred from the cyclotron to the hot-cell and trapped on a QMA cartridge (Waters SepPak Accell QMA cartridge carbonate). A mixture of kryptofix 2.2.2 (6.3 mg/mL), K₂C₂O₄ (1 mg/mL), and K₂CO₂ (0.1 mg/mL) in MeCN/H₂O (4:1) was applied to elute the radioactivity to the reaction vial. After azeotropic drying with MeCN (0.8 mL×3), 2-3 mg precursor 6 with 14 mg Cu(OTf)₂(py)₄ dissolved in DMA/n-BuOH (0.3 mL, v/v=2/1) was added to the residue, and the reaction mixture was heated at 110° C. for 10 min with ventilated needle. After dilution with 2.7 mL water, the mixture was proceeded with semi-preparative HPLC purification. The title compound was collected, concentrated, eluted and neutralized using the same procedure described above.

Step c—Synthesis of (R)-1-(4′-(fluoro-¹⁸F)[1,1′-biphenyl]-4-yl)-4-(4-furan-2-carbonyl)piperazin-1-yl)pyrrolidin-2-one ([¹⁸F]-II)

The title compound was synthesized by the same procedure like ([¹⁸F]-III), using 2-3 mg precursor 7. Their identities were confirmed by co-injection of corresponding compound in analytical HPLC with radiochemical purities greater than 99%.

### Example 7—MAGL Inhibitory Activity

Compounds were profiled for MAGL inhibitory activity by determining the enzymatic activity by following the hydrolysis of the natural substrate, 2-arachidonoylglycerol, resulting in arachidonic acid, which can be followed by mass spectrometry. This assay is hereinafter abbreviated “2-AG assay”.

The 2-AG assay was carried out in 384 well assay plates (PP, Greiner Cat #784201) in a total volume of 20 μL. Compound dilutions were made in 100% DMSO (VWR Chemicals 23500.297) in a polypropylene plate in 3-fold dilution steps to give a final concentration range in the assay from 12.5 μM to 0.8 pM. 0.25 μL compound dilutions (100% DMSO) were added to 9 μL MAGL in assay buffer (50 mM TRIS (GIBCO, 15567-027), 1 mM EDTA (Fluka, 03690-100ml), 0.01% (v/v) Tween. After shaking, the plate was incubated for 15 min at RT. To start the reaction, 10 μL 2-arachidonoylglycerol in assay buffer was added. The final concentrations in the assay was 50 pM MAGL and 8 μM 2-arachidonoylglyerol. After shaking and 30 min incubation at RT, the reaction was quenched by the addition of 40 μL of acetonitrile containing 4 μM of d8-arachidonic acid. The amount of arachidonic acid was traced by an online SPE system (Agilent Rapidfire) coupled to a triple quadrupole mass spectrometer (Agilent 6460). A C18 SPE cartridge (G9205A) was used in an acetonitrile/water liquid setup. The mass spectrometer was operated in negative electrospray mode following the mass transitions 303.1→259.1 for arachidonic acid and 311.1→267.0 for d8-arachidonic acid. The activity of the compounds was calculated based on the ratio of intensities [arachidonic acid/d8-arachidonic acid].

TABLE 1

| | | IC₅₀ (mouse, | IC₅₀ (human, |
|---|---|---|---|
| Compound | Structure | nM) | nM) |
| I | 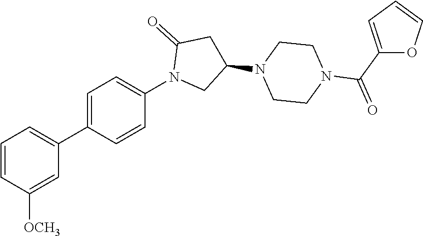 [SMILES: COc1cccc(-c2ccc(N3C[C@H](N4CCN(C(=O)c5ccco5)CC4)CC3=O)cc2)c1] | 4.3 | 6.5 |
| II | 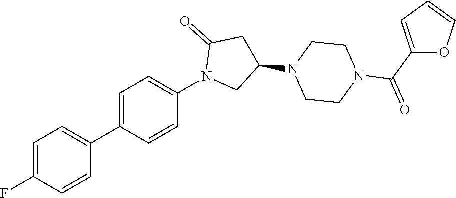 [SMILES: O=C(c1ccco1)N1CCN([C@@H]2CC(=O)N(c3ccc(-c4ccc(F)cc4)cc3)C2)CC1] | 8.8 | 20.0 |
| III | 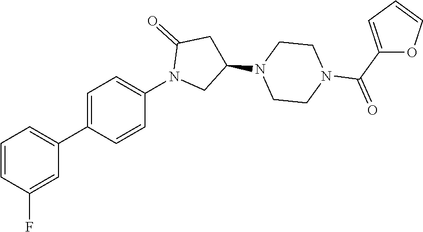 [SMILES: O=C(c1ccco1)N1CCN([C@@H]2CC(=O)N(c3ccc(-c4cccc(F)c4)cc3)C2)CC1] | 11.3 | 11.5 |

### Example 8—In Vitro Autoradiography

Frozen brain tissues of Wistar rats or mouse were cut in the thickness of 10 μm on a cryostat (Cryo-Star HM 560 MV; Microm, Thermo Scientific, Wilmington, DE), and stored at −20° C. before use. Before the experiments, the slices were thawed on ice for 10 min and subsequently immersed into aqueous 50 mM Tris buffer (pH 7.4, 30 mM HEPES, 1.2 mM MgCl₂, 110 mM NaCl, 5 mM KCl, 2.5 mM CaCl₂) containing 3% fatty acid free bovine serum albumin (BSA) at 0° C. for 10 min for precondition. Upon drying, incubation was carried out in a humidified chamber with the radiotracer at room temperature for 30 min. For blockade studies, 10 μM SAR127303 or 10 μM PF-06809247 were employed. The slices were decanted after incubation, and washing procedure was conducted. The slices were left to dry before attaching to phosphor imager plates (Fuji, Dielsdorf, Switzerland), and exposure was lasted for 60 min. The films were later scanned by BAS5000 reader (Fuji), and data analysis was performed using AIDA 4.50.010 software (Raytest Isotopenmessgerate GmbH, Straubenhardt, Germany).

### RESULTS

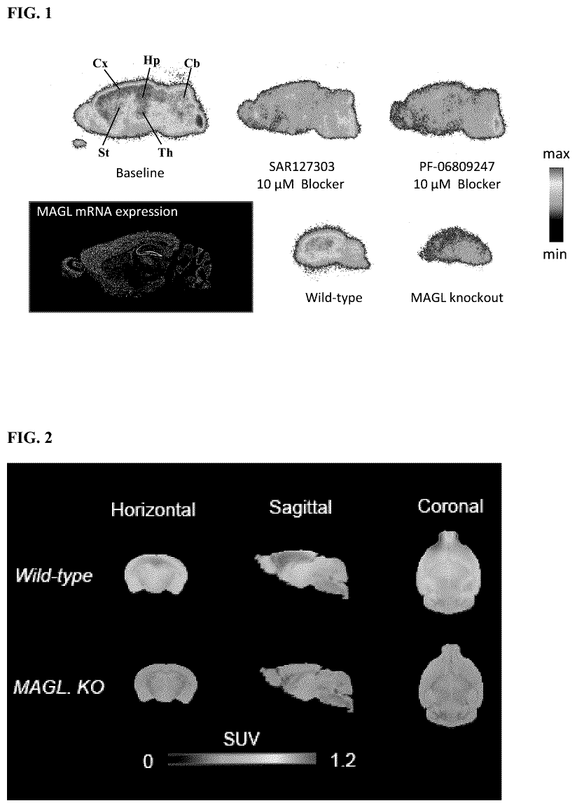

Incubation of [¹¹C]-I (˜1 nM) with mouse or rat brain slice resulted in a heterogeneous distribution of radioactive signal accumulation, which matched to the MAGL expression pattern in rodents (FIG. 1). Precisely, highest accumulation of [¹¹C]-I was observed in cortex, hippocampus and striatum where high levels of MAGL expression were reported (Dinh T P, Carpenter D, Leslie F M, et al. Brain monoglyceride lipase participating in endocannabinoid inactivation. Proc Natl Acad Sci. 2002;99:10819-10824). Two irreversible and potent MAGL inhibitors, SAR127303 and PF-06809247, were applied in blockade with a concentration of 10 μM to compete the specific binding with [¹¹C]-I on rat brain section. A substantial reduction and homogenous distribution of the radioactivity was observed in both cases, as well as simply incubated the probe on the MAGL KO brain section. These results confirm the high specificity and selectivity of [¹¹C]-I in vitro and suggest the reduced possibility of off-target binding in vivo.

Example 9—In Vivo PET Scans

The animal was anesthetized using isoflurane and placed in the PET/CT scanner (Super Argus, Sedecal, Madrid, Spain). The radiotracer was injected intravenously, and data was acquired at 1 min post-injection. The dynamic PET scan lasted 60 min for [¹¹C]-I, while it's prolonged to 90 min for [¹⁸F]-II and [¹⁸F]-III. The resulting data were reconstructed in user-defined time frames with a voxel size of 0.3875×0.3875×0.775 mm³. The regions of interest (ROI) were defined on an MRI T2 (W.Schiffer) template provided by PMOD v4.002 (PMOD Technologies, Zurich, Switzerland) to generate the corresponding time acitivity curves (TACs). The radioactivity accumulations in the whole brain and different regions were expressed as standardized uptake values (SUVs), which is the decay-corrected regional radioactivity normalized to the injected radioactivity and body weight.

### RESULTS

All three radiotracers were capable to cross the blood-brain barrier and reached the highest accumulation level within 5 min in mice brain. The representative whole brain TACs from [¹⁸F]-II and [¹⁸F]-III in MAGL KO and WT mice brains are decipted in FIG. 3. In the MAGL KO mice, strikingly faster clearance from the brain was observed compared to the WT mice, indicating their specific and selective binding in vivo. A profoundly increased brain uptake was achieved by [¹¹C]-I (SUVₘₐₓ˜1.32 at 1 min p.i), [¹⁸F]-II (SUVₘₐₓ˜1.56 at 1 min p.i) and [¹⁸F]-III (SUVₘₐₓ˜1.63 at 1 min p.i) in comparison with that of [¹⁸F]T-401 (SUVₘₐₓ˜0.7 reported in Hattori Y, Aoyama K, Maeda J, et al. Design, Synthesis, and Evaluation of (4R)-1-{3-[2-(¹⁸F)Fluoro-4-methylpyridin-3-yl]phenyl}-4-[4-(1,3-thiazol-2-ylcarbonyl)piperazin-1-yl]pyrrolidin-2-one ([¹⁸F]T-401) as a Novel Positron-Emission Tomography Imaging Agent for Monoacylglycerol Lipase. J Med Chem. 2019;62:2362-2375). The heterogeneous distributions of the probes in vivo were highly in accordance with the MAGL expression levels in the mouse brain. Representative PET images of [¹¹C]-I, which closely resembled to its in vitro autoradiograms, are shown in FIG. 2.

### Example 10—Drug Occupancy Study

To investigate the utility of [¹⁸F]-II, a drug occupancy study was conducted after validating the in vitro/vivo specificity. The potent and selective covalent MAGL inhibitor PF-06795071, originally developed for anti-inflammatory treatments, was applied for this study (McAllister LA, Butler C R, Mente S, et al. Discovery of Trifluoromethyl Glycol Carbamates as Potent and Selective Covalent Monoacylglycerol Lipase (MAGL) Inhibitors for Treatment of Neuroinflammation. J Med Chem. 2018;61:3008-3026). PF-06795071 was prepared as a transparent solution in a vehicle of DMSO/Cremophor/saline (v/v/v=5/5/90). The animal was treated with escalating doses of PF-06795071 (0.002, 0.01, 0.05, 0.2 and 2 mg/kg) 1 hour prior to the administration of [¹⁸F]-II (4.15-11.13 MBq, 6.84-13.19 nmol/kg). The in vivo PET scans and data reconstruction were carried out as described in Example 9. The SUVs from the whole brain TACs was averaged from 0-90 min to obtain SUV_{0-90 min} in receptor occupancy study as previously detailed by Krämer et al. (Krämer SD, Betzel T, Mu L, et al. Evaluation of ¹¹C-Me-NB1 as a Potential PET Radioligand for Measuring GluN2B-Containing NMDA Receptors, Drug Occupancy, and Receptor Cross Talk. J Nucl Med. 2018;59:698-703). The non-linear curve was fitted with GraphPad Prism Software (version 8.3.4, GraphPad Software Inc) for D₅₀ value.

### RESULTS

A dose-dependent reduction of [¹⁸F]-II accumulation in the mouse brain was observed with PF-06795071 ranging from 0.002 to 2 mg/kg. Averaged SUV_{0-90 min} from the whole brain TACs were then transferred into the percentage of target occupancy, and fitted into the saturation equation to determine the drug-target engagement (FIG. 4). The required dose for PF-06795071 to occupy 50% MAGL in the mouse brain was 0.034 mg/kg. This study demonstrated the utility of [¹⁸F]-II as a reversible MAGL PET tracer for visualizing drug-target engagement in vivo, as well as quantifying drug occupancy non-invasively. Target occupancy by a reversible MAGL PET tracer in rodents is unprecedented. These findings indicate that [¹⁸F]-II is a highly promising PET probe for visualizing MAGL non-invasively in vivo, and holds great potential for clinical translation.

### Example 11—X-ray Structure of Compound-II Bound to MAGL

Human MAGL protein with mutations Lys36Ala, Leu169Ser and Leu176Ser was concentrated to 10.8 mg/ml. Crystallization trials were performed in sitting drop vapor diffusion setups at 21° C. Crystals appeared within 2 days out of 0.1M MES pH 6.5, 6 to 13% PEG MME5K, 12% isopropanol. Crystals were soaked for 16 hours in crystallization solution supplemented with 10 mM of compound II. For data collection, crystals were flash cooled at 100K with 20% ethylene glycol added as cryo-protectant to the soaking solution. X-ray diffraction data were collected at a wavelength of 0.9999 Å using an Eiger2X 16M detector at the beamline X10SA of the Swiss Light Source (Villigen, Switzerland). Data have been processed with XDS (Kabsch W, XDS. Acta Cryst. D66, 125-132 (2010)) and scaled with SADABS (BRUKER). The crystals belong to space group C222₁ with cell axes of a=89.96 Å, b=127.45 Å, c=63.03 Åwith and diffract to a resolution of 1.65 Å. The structure was determined by molecular replacement with PHASER (McCoy A J, Grosse-Kunstleve R W, Adams P D, Winn M D, Storoni L C, & Read, R. J. Phaser crystallographic software. J Appl Cryst. 40, 658-674 (2007)) using the coordinates of PDB entry 3PE6 as search model (Schalk-Hihi C, Schubert C, Alexander R, Bayoumy S, Clemente J C, Deckman I, DesJarlais R L, Dzordzorme K C, Flores C M, Grasberger B, Kranz J K, Lewandowski F, Liu L, Ma H, Maguire D, Macielag M J, McDonnell M E, Mezzasalma Haarlander T, Miller R, Milligan C, Reynolds C, Kuo LC. Crystal structure of a soluble form of human monoglyceride lipase in complex with an inhibitor at 1.35 Å resolution. Protein Sci. 20(4), 670-83 (2011)).

### RESULTS

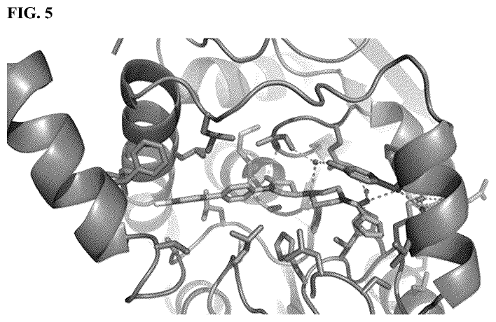

The complex structure of human MAGL with compound-II confirmed the reversible binding mechanism of compound II with the enzyme (FIG. 5). The pyrrolidinone oxygen locates in close proximity to the catalytic Ser122 and points towards the oxyanion hole forming hydrogen bonds with the main chain backbone amide from Met123 and Ala51.

1. A compound selected from the group consisting of 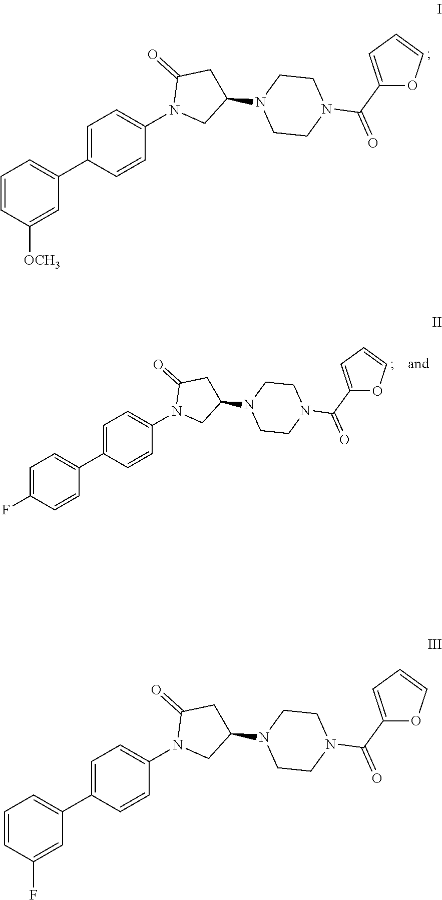 [SMILES: COc1cccc(-c2ccc(N3C[C@H](N4CCN(C(=O)c5ccco5)CC4)CC3=O)cc2)c1 ; O=C(c1ccco1)N1CCN([C@@H]2CC(=O)N(c3ccc(-c4ccc(F)cc4)cc3)C2)CC1 ; O=C(c1ccco1)N1CCN([C@@H]2CC(=O)N(c3ccc(-c4cccc(F)c4)cc3)C2)CC1]
or a pharmaceutically acceptable salt thereof, wherein said compound optionally comprises a radiolabel.

2. The compound according to claim 1, wherein said radiolabel is selected from ¹¹C and ¹⁸F.

3. The compound according to claim 2, selected from the group consisting of 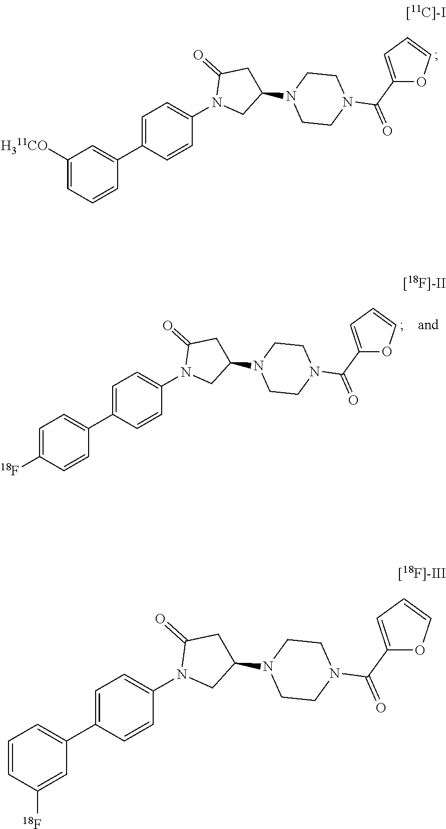
or a pharmaceutically acceptable salt thereof.

4. The compound according to claim 2, which is 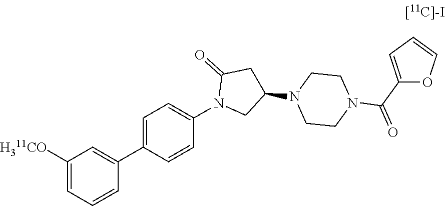
or a pharmaceutically acceptable salt thereof.

5. The compound according to claim 2, which is 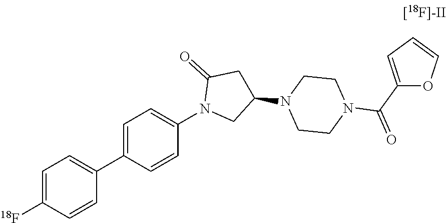 [SMILES: O=C(c1ccco1)N1CCN([C@@H]2CC(=O)N(c3ccc(-c4ccc([18F])cc4)cc3)C2)CC1]
or a pharmaceutically acceptable salt thereof.

6. The compound according to claim 2, which is 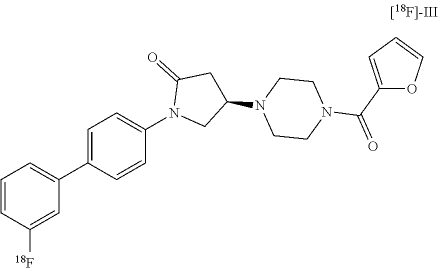 [SMILES: O=C(c1ccco1)N1CCN([C@@H]2CC(=O)N(c3ccc(-c4cccc([18F])c4)cc3)C2)CC1]
or a pharmaceutically acceptable salt thereof.

7. A pharmaceutical composition comprising a compound according to any one of claims 1 to 6, or a pharmaceutically acceptable salt thereof, and a pharmaceutically acceptable excipient.

8. A compound according to claim 1, for use as therapeutically active substance.

9. A method of diagnostic imaging of monoacylglycerol lipase (MAGL) in the brain of a mammal, comprising:
(a) administering to the mammal a detectable quantity of a radiolabeled compound according to any one of claims 1-6, or of a pharmaceutically acceptable salt thereof, or of a pharmaceutical composition according to claim 7; and
(b) detecting the radiolabeled compound when associated with MAGL.

10. A radiolabeled compound according to any one of claims 1 to 6, or a pharmaceutically acceptable salt thereof, or a pharmaceutical composition according to claim 7, for use in monoacylglycerol lipase (MAGL) occupancy studies.

11. A radiolabeled compound according to any one of claims 1 to 6, or a pharmaceutically acceptable salt thereof, or a pharmaceutical composition according to claim 7, for use in a method according to claim 9.

12. Use of a radiolabeled compound according to any one of claims 1 to 6, or of a pharmaceutically acceptable salt thereof, or of a pharmaceutical composition according to claim 7, in a method according to claim 9.

13. Use of a radiolabeled compound according to any one of claims 1 to 6, or of a pharmaceutically acceptable salt thereof, for the preparation of a medicament for the diagnostic imaging of monoacylglycerol lipase (MAGL) in the brain of a mammal.

14. A compound according to claim 1, for use in the treatment or prophylaxis of neuroinflammation, neurodegenerative diseases, pain, cancer, mental disorders, multiple sclerosis, Alzheimer's disease, Parkinson's disease, amyotrophic lateral sclerosis, traumatic brain injury, neurotoxicity, stroke, epilepsy, anxiety, migraine, depression, inflammatory bowel disease, abdominal pain, abdominal pain associated with irritable bowel syndrome and/or visceral pain.

15. The invention as hereinbefore described.
